(19) United States
(12) Patent Application Publication
(10) Pub. No.: US 2005/0257277 A1
(43) Pub. Date: Nov. 17, 2005

(54) Regulation of transcription with a cis-acting ribozyme

(76) Inventors: Xiaobin Lu, Germantown, MD (US); Boro Dropulic, Ellicott City, MD (US); Vladimir Slepushkin, Damascus, MD (US); Gwendolyn Binder, Montgomery Village, MD (US)

Correspondence Address: MORRISON & FOERSTER LLP, 3811 VALLEY CENTRE DRIVE, SUITE 500, SAN DIEGO, CA, 92130-2332, US

(21) Appl. No.: 10/847,728

(22) Filed: May 17, 2004

(51) Int. Cl. A01K 67/027; C12N 15/86

(52) U.S. Cl. 800/14; 435/456

(57) ABSTRACT

The present invention provides a recombinant transcription unit capable of producing an RNA transcript of a predetermined size comprising a regulatory sequence operably linked to a nucleotide sequence comprising a transcribed region such that the transcription of said transcribed region is controlled by said regulatory sequence. The transcribed region comprises a region that encodes for a viral sequence, and a non-coding region downstream of the region encoding for said viral sequence, wherein the non-coding region comprises a nucleotide sequence encoding a cis-acting ribozyme. Methods of using the recombinant transcription unit, and cells containing vectors comprising the recombinant transcription unit are also disclosed.

### CROSS-REFERENCE TO RELATED APPLICATIONS

This application is related to U.S. patent application Ser. No. 10/627,940, filed Jul. 25, 2003, which is hereby incorporated by reference as if fully set forth.

### TECHNICAL FIELD

The present invention is in the field of molecular biology and recombinant DNA technology. More specifically, recombinant DNA constructs that can produce a transcript capable of self cleavage to limit the size and thus content of the transcript are provided. The invention thus provides methods for the preparation of such constructs as well as methods for the use of such constructs to produce a transcript of a predetermined size. The invention is particularly advantageous to limit the extent of transcriptional read-through following a polyadenylation signal. The invention can be used to transcribe and/or translate any known nucleotide sequence.

### BACKGROUND ART

Because of the circular nature of a DNA or RNA plasmid or vector, and the presence on the plasmid of often more than one transcription unit comprising a promoter and a nucleotide sequence to be transcribed, transcription from any one promoter can potentially result in a longer polycistronic messenger RNA than desired. If a nucleotide sequence downstream of a promoter encodes, for example, a protein, the expression levels of the protein will be affected. In addition, the improper message can serve as a potential substrate for generation of replication competent viruses, for example, through RNA-based recombination events during reverse transcription.

Thus, it would be advantageous to utilize a plasmid wherein transcriptional read-through is prevented or reduced.

Problems relating to transcriptional read-through often arise when making transgenic animals. Transgenic animals have inserted into their genome a gene or genes in which the effect(s) of the gene or genes is intended to be studied. Often the first gene is expressed, then at a different point in time the second gene is expressed which has an effect on the first gene. The genes are placed in a vector in order to facilitate insertion into the animal's cells and genome. Often the first gene is placed downstream of the second gene on the vector. Even though the first gene may have a cleavage signal as part of its nucleotide sequence, often upon transcription of the first gene the RNA polymerase reads through the cleavage signal and transcribes the second gene. If this type of read-through transcription occurs, both the first and second genes may be transcribed, preventing the effects of the first gene to be studied in the absence of the expression of the second gene.

Thus, it would be advantageous to create a plasmid in which read-through transcription between the first and second gene is prevented.

Lastly, when trying to produce large amounts of retroviral vectors for the purpose of utilizing these vectors as research tools and/or disease prevention and/or treatment vehicles low yields of vectors often result. These low yields can be due to the competition/interference between the three different vectors that contain all the necessary nucleotide sequences required to package the virus. Three different vectors are used to keep the retrovirus “in check”, preventing any one vector from obtaining all the necessary components to become a replication competent vector. In addition, it is often costly and time consuming to make the three different vectors.

Thus, it would be advantageous to have a retroviral vector production system comprising only two vectors wherein neither vector is capable of becoming a replication competent vector. In addition, two-plasmid production reduces plasmid production costs, which can be significant at the commercial scale. In order to have a two-vector system, components from two of the three vectors would have to be combined into one vector. Therefore, a way to ensure that there is no read-through transcription from one set of components to another set of components that were previously separated on different vecotrs is to insert a ribozyme between the two sets of components, thus for all functional purposes making a single plasmid containing two transcriptional units that behave effectively as two separate plasmids in terms of safety, while maintaining the cost and efficacy advantages of a two-plasmid production system. Hence, a two-plasmid system would result in higher yields of retroviral vectors, for example, and reduce the overall cost of maintaining a three-plasmid system.

Ribozymes are small RNAs that contain catalytic activity. These small RNAs range in size from 40 nucleotides to 2600 nucleotides, depending upon the nature of the ribozyme. Ribozymes are naturally occurring, and are thought to be the earliest enzymes catalyzing chemical reactions before proteins had formed.

There exist cis-acting and trans-acting ribozymes. Cis-acting ribozymes can act on a target RNA that is adjacent, proximal, or far away from its location. Hammerhead, Hepatitis delta virus (HDV), hairpin, Varkud satellite (VS), Group I intron, and Group II intron are examples of various types of cis-acting ribozymes (Doudna, J. and Cech, T., Nature, 418: 222-228 (2002)).

Ribozyme cleavage is site-specific and is mediated by hydrogen bonding between complementary bases at target regions. The catalytic unit of the ribozyme mediates cleavage by facilitating atom replacement, which causes a break in the target RNA backbone.

According to current knowledge, ribozyme reactions are irreversible in their natural setting. Thus, ribozymes are able to effect permanent cleavage at distinct sites.

### DISCLOSURE OF THE INVENTION

The invention provides a method for making a transcription unit which produces an RNA transcript of a predetermined size by introduction of a sequence encoding a cis-acting ribozyme into the non-coding region of said transcription unit such that transcription of the unit into an RNA molecule includes generation of the ribozyme. Upon production of the ribozyme, it can act in cis to cleave the RNA transcript at the ribozymes recognition site for cleavage to limit the size of the RNA. The invention thus provides recombinant DNA constructs that can be transcribed to produce an RNA transcript capable of self cleavage to limit its size. This provides the ability to control the content of the transcript to permit regulation of undesirable effects such as transcriptional read-through.

The transcription units of the invention are recombinant DNA constructs comprising sequences that regulate transcription, such as one or more promoter, as well as the sequences that may be transcribed under the control of the promoter. The sequences that may be transcribed may encode a polypeptide of interest or not encode any polypeptide. The transcription unit can also contain non-coding sequences such as a 3′ untranslated sequence, a polyadenylation signal, a pause site, a strong pause site, or a near upstream (NUE) sequence, for example.

While the invention provides for the insertion of a sequence encoding a cis-acting ribozyme into a transcription unit by introduction of the sequence into either coding or non-coding sequences, this may be accomplished by a variety of means, including insertion into a coding or non-coding sequence already operably linked to regulatory sequences or insertion into a coding or non-coding sequence prior to its operable linkage to regulatory sequences. The sequence encoding a cis-acting ribozyme need only be downstream of, or be 3′ from, the regulatory sequences of the transcription unit such that the ribozyme may be produced upon transcription of the unit to produce RNA. The sequence encoding a cis-acting ribozyme may even be downstream of a cleavage signal, for example, a polyadenylation signal present in the case of a eukaryotic transcription unit. The invention also comprises, however, prokaryotic transcription units that do not comprise a polyadenylation signal.

The constructs of the invention may be considered recombinant in that they are not naturally occurring in nature. Preferably, the sequence encoding a cis-acting ribozyme is introduced into a heterologous coding or non-coding sequence with which the ribozyme is normally not found in nature.

The invention thus permits the use of a sequence encoding a cis-acting ribozyme to regulate the size of RNA transcripts encoding a variety of polypeptides. In one aspect of the invention, the polypeptides are those of a virus, such as a lentivirus or HIV. In another aspect of the invention, the polypeptides are essential to virus replication and/or spread, such as a viral envelope protein. However, the invention is not limited by the type of polypeptide that is encoded. Indeed, the invention may be practiced in cases of transcription units that do not encode any polypeptide if so desired.

Similarly, the invention is not limited by the source, identity, or type of cis-acting ribozyme used. A variety of such ribozymes may be used, and non-limiting examples include Hammerhead, Hepatitis delta virus, Hairpin, Varkud satellite, group I intron, and group II intron. Cis-acting ribozymes are described in D. B. McKay and J. E. Wedekind, The RNA World 265-286 (R. F. Gesteland, T. R. Cech, J. F. Atkins, eds., CSH Laboratory Press 1999). Hairpin and hammerhead ribozymes are described in Burke, J. M., Biochemical Soc. Trans., 30:1116-1119 (2002). A preferred cis-acting ribozyme for the practice of the invention is that of the satellite RNA of tobacco ringspot virus (sTobRV). The satellite RNA of tobacco ringspot virus is described in Haseloff, J. and Gerlach, W. L., Gene, 82:43-52 (1989).

The invention is particularly advantageous as a method of limiting the size of an RNA transcript produced from a transcription unit. As such, it is somewhat functionally similar to a polyadenylation signal in a eukaryotic transcription unit in that the ribozyme, like a polyadenylation signal, results in the cleavage of an RNA transcript and thus limits its size. This occurs in a transcription unit of the invention when it is placed under conditions wherein the sequence encoding a cis-acting ribozyme is transcribed. As such, the invention may be used to provide a polyadenylation signal-like cleavage function in a prokaryotic transcription unit.

Where the sequence encoding a cis-acting ribozyme is downstream of a cleavage signal, for example a polyadenylation signal, cleavage directed by the polyadenylation signal may prevent transciption of the sequence encoding the ribozyme. But in such cases, the sequence encoding a cis-acting ribozyme may be considered a secondary cleavage signal in the transcription unit to ensure cleavage in the event that cleavage directed by the polyadenylation signal does not occur.

The ability to limit the size of an RNA transcript is particularly advantageous in cases of multicistronic DNA constructs where transcriptional read-through is undesirable. The ability to limit the size of an RNA transcript is also advantageous in cases where a longer transcript increases the likelihood of recombination with another sequence.

The presence of a nucleotide sequence encoding a ribozyme that is located in between two or more transcription units would be useful in preventing transcriptional read-through. In addition, the presence of a nucleotide sequence encoding a ribozyme that is located at the end of a single transcription unit would also be advantageous in that the ribozyme can act as a back-up in case the cleavage signal that exists in the transcription unit is not working properly.

One benefit of cis-acting ribozymes for separation of transcriptional units is to improve the safety of a two plasmid viral vector production system by reducing the probability of recombination resulting in a replication competent virus. In order to have a two vector system, components from two of the three vectors would have to be combined into one vector. Therefore, a way to ensure that there is no read-through transcription from one set of components to another set of components that were previously separated on different vectors, is to insert a ribozyme between the two sets of components. A two vector system would have less interference between vectors due to there being one less vector, resulting in a more efficient production of the retroviral vectors.

Two plasmid viral vector production in the large scale is less expensive and yields higher vector titers than when using a three plasmid viral vector production system. Therefore, incorporating a cis-acting ribozyme into a two plasmid viral vector production system can represent a significant advantage in manufacture.

In three plasmid viral vector production, vector containing payload is placed on one plasmid (the vector plasmid), structural genes on a second plasmid (helper plasmid) and non-structural genes on a third plasmid (another helper plasmid). Alternatively, structural and non-structural genes are placed on the second plasmid and the envelope gene is placed on the third plasmid. Incorporating a cis-acting ribozyme onto the second plasmid functionally allows a single plasmid to act as a second and third plasmid in one. Thus, by inserting a cis-acting ribozyme between, for example, the structural genes and the non-structural genes, not only is the risk of read-through transcription prevented, but incorporation of a cis-acting ribozyme allows the two helper plasmids to be consolidated into a single helper plasmid.

Specifically, incorporation of the cis-acting ribozyme results in immediate cleavage of RNA transcripts separated by the ribozyme. This ensures that a single recombination event between the RNA transcribed from the vector plasmid and with one of the RNAs from the helper plasmid containing the cis-ribozyme cannot result in a replication competent virus. Such a recombination is likely to occur during the process of reverse transcription, when the polymerase commonly jumps between the RNAs encapsulated within the particle creating a complementary DNA sequence that contains genes from both RNAs. Thus, incorporation of a cis-acting ribozyme in a single plasmid dividing structural and non-structural genes offers equivalent safety in a two plasmid system to that of a three plasmid system, and allows the added benefit of higher titers and lower production costs at the large scale.

The payload can be, for example, an antisense molecule, a RNA decoy, a transdominant mutant, a toxin, a single-chain antibody (scAb) directed to a viral structural protein, a siRNA, or a ribozyme.

A structural gene can be, for example, gag, a gag-pol precursor, pro, reverse transcriptase (RT), integrase (In) or env. A non-structural gene can be, for example, tat, rev, nef, vpr, vpu, or vif.

A therapeutic use of cis-acting ribozymes in vivo permits a cell to be co-transduced with a helper plasmid and a vector plasmid, so that the helper plasmid transcription is inducible and results in expression and propagation of the vector plasmid, without high risk of replication competent virus generation. For example, the cells may be transduced with a helper SIN vector that cannot replicate on its own, but when specifically activated by the promoter, can propagate the mobilizable vector plasmid containing functional LTRs. The helper SIN vector can contain the necessary structural and non-structural genes separated by a sequence encoding a cis-ribozyme. There are several reasons that this is an improvement over using two helper plasmids. First, transduction of the target cells with a given target ratio of helper plasmid to vector is more easily obtainable given two plasmids instead of three. Second, control of helper plasmid expression is more easily regulated if helper genes are each expressed under the same inducible promoter.

Antiviral, or antivector, antisense or ribozymes may also be retained on the helper plasmid component of the vector packaging system. This may serve as a safety mechanism for vector packaging to reduce potential generation of a replication competent lentivirus (RCL). The antiviral or antivector sequence would be targeted to a sequence present in the vector genome, but not be expressed as a separate transcript capable of blocking productive vector packaging in the cell. The antiviral or antivector sequence would be intended to block propagation of vector particles containing a copy of the helper plasmid with a copy of the vector genome, instead of the vector genome RNA duplex normally found in vector particles. Non-specific packaging of appropriately sized nucleic acid sequences not containing a packaging signal have been previously described, and is known to occur at low frequencies. Therefore, this design may have important implications for the safety of retroviral production.

Cis-acting ribozymes may also facilitate the generation of transgenic animals, for example, mice. Similarly as above, the cis-acting ribozyme may be used to separate transcripts expressed within the transgenic construct to be introduced into a mouse. For example, the use of a ribozyme inserted into the plasmid between the end of the first gene and the promoter that transcribes the second gene could be advantageous. As a non-limiting example this is useful when the gene being expressed for study is a dominant negative of the gene of interest. Expressed on the same transcript is a second gene that can convert the dominant negative transgene to a functional gene. Efficient separation of the transcripts would be critical to successful analysis of the mouse phenotype, since if read through occurred into the converting gene from the dominant negative, the phenotype would be masked. Pause sites, strong pause sites, and poly-A signals have historically been used to achieve this goal. However, as the results in the provided examples show, these stop signals insufficiently prevent read through from occurring at low levels. Therefore, using a cis-acting ribozyme in addition to, or in place of, these sites, may ensure greater success when creating a transgenic mouse to determine gene function.

One aspect of the invention is a method of preparing a recombinant transcription unit capable of producing an RNA transcript of a predetermined size comprising, operably linking a regulatory sequence and a nucleotide sequence comprising a transcribed region such that transcription of the transcribed region is controlled by the regulatory sequence, wherein the transcribed region comprises a region that encodes a viral sequence and a non-coding region downstream of the region encoding for the viral sequence, and wherein the non-coding region comprises a nucleotide sequence encoding a cis-acting ribozyme.

The non-coding region can further comprises a nucleotide sequence encoding a cleavage signal upstream of the nucleotide sequence encoding a cis-acting ribozyme.

The viral sequence can be, for example, a viral protein. The viral protein, can be, for example, a protein encoded by a lentivirus or a viral envelope protein. The viral protein can be, for example, VSV-G, gag, pol, tat, or rev, or any combination of VSV-G, gag, pol, tat, and rev.

The viral sequence can further comprise a nucleotide sequence encoding an antiviral agent that is either upstream or downstream of the nucleotide sequence encoding the viral protein. The antiviral agent can be, for example, an antisense molecule or a ribozyme.

Another aspect of the invention is a host cell comprising a recombinant transcription unit capable of producing an RNA transcript of a predetermined size, wherein the transcription unit comprises a regulatory sequence operably linked to a nucleotide sequence comprising a transcribed region such that the transcription of the transcribed region is controlled by the regulatory sequence, wherein the transcribed region comprises a region that encodes for a viral sequence, and a non-coding region downstream of the region encoding for the viral sequence, and wherein the non-coding region comprises a nucleotide sequence encoding a cis-acting ribozyme. The non-coding region can further comprise a nucleotide sequence encoding a cleavage signal upstream of the nucleotide sequence encoding the cis-acting ribozyme

Yet another aspect of the invention is a recombinant transcription unit capable of producing an RNA transcript of a predetermined size comprising a regulatory sequence operably linked to a nucleotide sequence comprising a transcribed region encoding a viral sequence and a non-coding region downstream of the region encoding for said viral sequence, wherein the non-coding region comprises a nucleotide sequence encoding a cis-acting ribozyme. The non-coding region can further comprise a nucleotide sequence encoding a cleavage signal upstream of the nucleotide sequence encoding the cis-acting ribozyme.

The viral sequence in the transcription unit can be, for example, a viral protein. The viral protein, can be, for example, a protein encoded by a lentivirus or a viral envelope protein. The viral protein can be, for example, VSV-G, gag, pol, tat, or rev, or any combination of VSV-G, gag, pol, tat, and rev.

The viral sequence can further comprise a nucleotide sequence encoding an antiviral agent that is either upstream or downstream of the nucleotide sequence encoding the viral protein. The antiviral agent can be, for example, an antisense molecule or a ribozyme.

Another aspect of the invention is a method of limiting the size of an RNA transcript produced from a transcription unit, the method comprising, inducing transcription of a transcription unit comprising a regulatory sequence operably linked to a nucleotide sequence comprising a transcribed region such that the transcription of the transcribed region is controlled by the regulatory sequence, wherein the transcribed region comprises a region that encodes for a viral sequence, and a non-coding region downstream of the region encoding for the viral sequence, wherein the non-coding region comprises a nucleotide sequence encoding a cis-acting ribozyme, and wherein the transcription unit produces a transcript under conditions wherein the sequence encoding the cis-acting ribozyme is transcribed and cleaves the transcript in cis. The non-coding region can further comprise a nucleotide sequence encoding a cleavage signal upstream of the nucleotide sequence encoding the cis-acting ribozyme.

The viral sequence of the method can be, for example, a viral protein. The viral protein, can be, for example, a protein encoded by a lentivirus or a viral envelope protein. The viral protein can be, for example, VSV-G, gag, pol, tat, or rev, or any combination of VSV-G, gag, pol, tat, and rev.

The viral sequence can further comprise a nucleotide sequence encoding an antiviral agent that is either upstream or downstream of the nucleotide sequence encoding the viral protein. The antiviral agent can be, for example, an antisense molecule or a ribozyme.

Another aspect of the invention is a vector comprising, a first transcription unit capable of producing a first RNA transcript of a predetermined size, wherein the first transcription unit comprises a first promoter operably linked to a nucleotide sequence comprising a transcribed region such that the transcription of the transcribed region is controlled by the first promoter, wherein the transcribed region comprises a region that encodes for a first gene, and a first non-coding region downstream of the region encoding for the first gene, wherein the first non-coding region comprises a nucleotide sequence encoding a cis-acting ribozyme, and a second transcription unit capable of producing a second RNA transcript of a predetermined size, wherein the second transcription unit comprises a second promoter operably linked to a nucleotide sequence comprising a transcribed region such that the transcription of the transcribed region is controlled by the second promoter, wherein the transcribed region comprises a region that encodes for a second gene, and a second non-coding region downstream of the region encoding for the second gene, and wherein the second non-coding region comprises a nucleotide sequence encoding a cis-acting ribozyme. In addition, the first gene, second gene, or both can have at their carboxy termini a cleavage signal.

The vector can have, for example, a first promoter that is constitutive and a second promoter that is inducible. The vector can have, for example, a first gene that is a dominant negative transgene and the second gene that is a gene that when expressed the expression product can convert the dominant negative transgene into a functional gene. The first gene can be, for example, a proenzyme and the second gene's expression product converts the proenzyme to an active enzyme. The first gene can encode for, for example, a protein in which at least one amino acid of the protein is capable of being phosphorylated and the second gene can encode for a kinase capable of phosphorylating the amino acid of the protein. Alternatively, the first gene can encode for a first protein which comprises at least one phosphorylated amino acid and the second gene can encode for a protein phosphatase capable of dephosphorylating the amino acid of the first protein.

Yet another aspect of the invention is a host cell comprising a vector that comprises a first transcription unit capable of producing a first RNA transcript of a predetermined size, wherein the first transcription unit comprises a first promoter operably linked to a nucleotide sequence comprising a transcribed region such that the transcription of the transcribed region is controlled by the first promoter, wherein the transcribed region comprises a region that encodes for a first gene, and a first non-coding region downstream of the region encoding for the first gene, wherein the first non-coding region comprises a nucleotide sequence encoding a cis-acting ribozyme, and a second transcription unit capable of producing a second RNA transcript of a predetermined size, wherein the second transcription unit comprises a second promoter operably linked to a nucleotide sequence comprising a transcribed region such that the transcription of the transcribed region is controlled by the second promoter, wherein the transcribed region comprises a region that encodes for a second gene, and a second non-coding region downstream of the region encoding for the second gene, and wherein the second non-coding region comprises a nucleotide sequence encoding a cis-acting ribozyme. In addition, the first gene, second gene, or both can have at their carboxy termini a cleavage signal.

Another aspect of the invention is a method of making a transgenic animal comprising inserting into the genome of the animal a vector comprising, a first transcription unit capable of producing a first RNA transcript of a predetermined size, wherein the first transcription unit comprises a first promoter operably linked to a nucleotide sequence comprising a transcribed region such that the transcription of the transcribed region is controlled by the first promoter, wherein the transcribed region comprises a region that encodes for a first gene, and a first non-coding region downstream of the region encoding for the first gene, wherein the first non-coding region comprises a nucleotide sequence encoding a cis-acting ribozyme, and a second transcription unit capable of producing a second RNA transcript of a predetermined size, wherein the second transcription unit comprises a second promoter operably linked to a nucleotide sequence comprising a transcribed region such that the transcription of the transcribed region is controlled by the second promoter, wherein the transcribed region comprises a region that encodes for a second gene, and a second non-coding region downstream of the region encoding for the second gene, wherein the second non-coding region comprises a nucleotide sequence encoding a cis-acting ribozyme. In addition, the first gene, second gene, or both can have at their carboxy termini a cleavage signal.

The vector of the method can be, for example, inserted into the genome of the germline of an animal, inserted into the genome of an unfertilized or fertilized egg of an animal, inserted into the genome of an embryo of an animal, or inserted into the genome of a cell located in the uterus of said animal.

Another aspect of the invention is a transgenic non-human animal comprising a vector which comprises, a first transcription unit capable of producing a first RNA transcript of a predetermined size, wherein the first transcription unit comprises a first promoter operably linked to a nucleotide sequence comprising a transcribed region such that the transcription of the transcribed region is controlled by the first promoter, wherein the transcribed region comprises a region that encodes for a first gene, and a first non-coding region downstream of the region encoding for the first gene, wherein the first non-coding region comprises a nucleotide sequence encoding a cis-acting ribozyme, and a second transcription unit capable of producing a second RNA transcript of a predetermined size, wherein the second transcription unit comprises a second promoter operably linked to a nucleotide sequence comprising a transcribed region such that the transcription of the transcribed region is controlled by the second promoter, wherein the transcribed region comprises a region that encodes for a second gene, and a second non-coding region downstream of the region encoding for the second gene, wherein the second non-coding region comprises a nucleotide sequence encoding a cis-acting ribozyme. In addition, the first gene, second gene, or both can have at their carboxy termini a cleavage signal.

Yet another aspect of the invention is a two vector retrovirus production system comprising, a first vector comprising a nucleotide sequence encoding a payload and a first promoter that controls transcription of the payload, and a second vector comprising a nucleotide sequence encoding a structural gene and a second promoter which controls transcription of the structural gene, and a nucleotide sequence encoding a non-structural gene and a third promoter which controls transcription of the non-structural gene, wherein the nucleotide sequence encoding the structural gene and the nucleotide sequence encoding the non-structural gene are separated by a nucleotide sequence encoding a cis-acting ribozyme.

Another aspect of the invention is a two vector retrovirus production system comprising, a first vector comprising a nucleotide sequence encoding a payload and a first promoter that controls transcription of the payload, and a second vector comprising a nucleotide sequence encoding a structural gene and a second promoter that controls transcription of the structural gene, a nucleotide sequence encoding a non-structural gene and a third promoter that controls transcription of the non-structural gene, and a nucleotide sequence encoding an envelope gene and a fourth promoter that controls transcription of the envelope gene, wherein each of the nucleotide sequences encoding the three genes are separated by a nucleotide sequence encoding a cis-ribozyme.

Yet another aspect of the invention is a method of producing a retrovirus comprising contacting a cell with a two vector retrovirus production system comprising, a first vector comprising a nucleotide sequence encoding a payload and a first promoter that controls transcription of the payload, and a second vector comprising a nucleotide sequence encoding a structural gene and a second promoter that controls transcription of the structural gene, a nucleotide sequence encoding a non-structural gene and a third promoter that controls transcription of the non-structural gene, wherein the nucleotide sequence encoding the structural gene and the nucleotide sequence encoding the non-structural gene are separated by a nucleotide sequence encoding a cis-acting ribozyme.

Another aspect of the invention is a method of producing a retrovirus comprising contacting a cell with a two vector retrovirus production system comprising, a first vector comprising a nucleotide sequence encoding a payload and a first promoter that controls transcription of the payload, and a second vector comprising a nucleotide sequence encoding a structural gene and a second promoter that controls transcription of the structural gene, a nucleotide sequence encoding a non-structural gene and a third promoter that controls transcription of the non-structural gene, and a nucleotide sequence encoding an envelope gene and a fourth promoter that controls transcription of the envelope gene, wherein each of the nucleotide sequences encoding the three genes are separated by a nucleotide sequence encoding a cis-ribozyme.

### BRIEF DESCRIPTION OF THE DRAWINGS

FIG. 1. Diagrammatic representation of the packaging plasmid for two HIV-based vectors, one containing (pVRX577) and one not containing (pVRX170) the ribozyme derived from the satellite RNA of the Tobacco Ringspot Virus. The sequence of the inserted ribozyme is expanded below the plasmid, and the black arrow notes the site of cleavage. The ribozyme is preceded in the plasmid by poly-A and transcriptional pause sites. The inclusion of such sites is not required for activity of the ribozyme.

FIG. 2. (A) Illustration of a read through virus packaging plasmid (VIRPAC) RNA containing a cis-acting ribozyme and in vitro validation of ribozyme function. A 1300-base region of VIRPAC without a native cis-acting ribozyme (− cis-RZ) (top of FIG. 2A) and VIRPAC with a cis-acting ribozyme (+ cis-Rz) (bottom of FIG. 2A) were amplified by PCR using primers containing a T7 promoter. The resulting DNA was then transcribed in vitro. (B) 2 μl of transcribed RNA at a concentration of about 1 μg/μl was added to 2 μl of RT-PCR buffer, then 2 μl (2 μg) was loaded onto the gel for visualization. Cleavage occurs rapidly, as no difference between 5, 10, 20, and 60 minutes of incubation in buffer prior to gel loading was observed (data not shown).

FIG. 3. Illustration of helper region containing the cis-acting ribozyme, and the location of PCR primers A, B and D for detection of transcriptional read through. Shown below the illustration is a schematic demonstrating the protocol for determination of assay sensitivity. Also represented is the process for manufacture of the lentiviral vector.

FIG. 4. Visualization of reverse transcription (RT) PCR products resulting from a positive control spike dilution series. The concentration of the spiked RNA control per μg of cellular RNA, DNA or per cell is represented above each lane.

FIG. 5. RT-PCR assay for the detection of transcriptional read through in helper constructs. Primer pair A/B detects plasmid RNA without read through, and primer pair A/D detects plasma RNA only in the event of read through (refer to schematic in FIG. 3). The sensitivity of this assay is 45 copies per μg of cellular RNA, and in vitro transcribed RNA from VRX170 was used as the positive control and standard. In 100% of 13 assays, when 45 spiked copies of control RNA were added to the reaction, message was detected. 15 spiked copies were detected 8 of 13 times, and in one experiment, a 5-copy spike was detected.

FIG. 6. No detection of transcriptional read through by RT-PCR in a helper construct containing a cis-acting ribozyme (VRX577). The experiment was conducted exactly as that presented above in FIG. 5. Each experiment represents an independent transfection of 293F cells with VRX577 and VRX496. Each reaction was run in triplicate (a, b, c. The sensitivity of the reaction was 45 copies/μg of cellular RNA.

FIG. 7. Inclusion of a cis-acting ribozyme does not affect the titers of vector produced. Helper plasmids containing (VRX577) and not containing (VRX170) a cis-acting ribozyme to separate transcriptional units were used to produce an HIV-based lentivirus vector (VRX496) in triplicate by cotransfection of vector and helper plasmids in 293F cells. 2-3 days later, vector-containing supernatants were collected and titered on HeLa-tat cells. As a control, cells were transfected with vector only. Titers are shown on the left as transducing units (TU) per ml of media.

### MODES OF CARRYING OUT THE INVENTION

A transcription unit of the invention comprises a regulatory sequence operably linked to a nucleotide sequence comprising a transcribed region such that the transcription of said transcribed region is controlled by the regulatory sequence. The transcribed region comprises a region that encodes for a viral sequence, and a non-coding region downstream of the region encoding for the viral sequence, wherein the non-coding region comprises a nucleotide sequence encoding a cis-acting ribozyme.

Example 1 provides two non-limiting examples of transcriptional units. Transcriptional unit one comprises: the cytomegalovirus (CMV) promoter, including the HIV-1 GagPol and TatRev genes, and terminating at the end of the bovine growth hormone polyadenylation (poly-A) signal. A ribozyme is placed in the vector immediately after the poly-A signal. Transcriptional unit two comprises: the elongation factor (EF) promoter, including the VSV-G gene, and terminating at the end of the SV40 poly-A site. A ribozyme could be placed, if desired, in the vector immediately after the poly-A site.

Numerous types of transcription units can be made. One skilled in the art could easily construct various types of transcription units using well known methods. Transcription units comprising, for example, any promoter, and any sequence or gene, with any termination signal can be made. In addition, a ribozyme can then be added to the vector immediately after the termination signal. If the transcription unit is going to be used, for example, to produce a protein in a bacterial culture, the unit would comprise a promoter, a gene, and a cis-ribozyme.

Any peptide can be used in the transcription unit of the invention as long as its activity is independent of cis-ribozyme function.

Cleavage sites of many ribozymes are well known in the art. One of skill in the art would easily be able to choose a ribozyme and insert it into the transcription unit of the invention.

The ability to inhibit read-through transcription can be used in other viral systems besides HIV. Specifically, the transcription unit permits any vector production that uses three plasmids to be reduced to two plasmids.

The Production of Viral Particles

The constructs and cells of the invention can be designed to provide the necessary factors to produce any viral particle containing a particular viral nucleic acid of interest. The viral nucleic acid can be replication deficient and derived from a naturally occurring virus without removal or loss of the endogenous “packaging signal”. In addition, the viral nucleic acid can be derived from HIV-1. HIV-1 derived viral nucleic acids may be produced by the pNL4-3 HIV-1 molecular clone which is a wild-type strain, available from the AIDS Research and Reference Reagent Program Catalog through the National Institutes of Health (see, also, Adachi, et al., J. Virol., 59, 284-291 (1986)). The cells may be viewed and used as “packaging cells” for the viral nucleic acid, which may be separately introduced into the cell, because they produce all the components necessary to package the viral nucleic acid into infectious viral particles.

Packaging cells can express from a coding sequence of interest at least a viral envelope protein, or equivalent (such as a mutant, fusion, or truncated form thereof) or heterologous form thereof, when the viral nucleic acid provides all other components. Examples of envelope proteins are those encoded by sequences endogenous to the viral nucleic acid in its natural form (i.e. that is normally used in the packaging of the virus from which the viral nucleic acid is derived) or heterologous to the viral nucleic acid. A variety of envelope proteins may be expressed in the practice of this aspect of the invention, including proteins to alter the target cell specificity of a packaged viral particle or alternate envelope proteins that result in pseudotyped viral particles. Examples of heterologous envelope proteins for use with HIV-1 derived viral nucleic acids include the VSV G protein, the Mokola virus G protein, and the HIV-2 envelope protein.

Alternatively, the cells can provide at least a viral envelope protein and one or more than one protein necessary for expression of packaging components from the viral nucleic acid to be packaged. A non-limiting example is cells which provide both an envelope protein as well as a cognate tat protein, or one or more than one other protein required in trans, to package a retroviral nucleic acid (e.g. cells that provide a VSV G protein and an HIV-1 tat protein to package an HIV-1 derived vector). Examples of additional proteins required in trans include those encoded by gag, pol, and rev sequences.

The viral nucleic acid of interest to be packaged can lack the ability to express or encode one or more than one viral accessory protein sequences (such as, but not limited to, Vif, Vpu, Vpr or Nef, or combinations or fragments thereof) that would make the nucleic acid pathogenic or possibly pathogenic. This may be achieved by removal of the corresponding coding sequences or mutating them to prevent their expression at the transcription or translation level. Such proteins, to the extent that they are necessary for packaging, would be supplied by the packaging cell either via the constructs of the invention or by an additional nucleic acid construct.

The Production of a Transgenic Animal

The general procedure for producing a trangenic mouse is described in Brinster, R. L., et al., Cell, 27:223 (1981).

Transgenic single-cell organisms, plants, and animals can be produced readily by several different methods known to one of skill in the art. These modified organisms contain one or more copies of a cloned gene integrated into the genome.

The transgenic technique can be used to introduce a normal copy of a gene into a mutant organism, thereby identifying a cloned DNA corresponding to a mutation-defined gene. It also is used to study sequences necessary for gene expression, to develop mouse models of dominant forms of human diseases, to modify plants, and to investigate the relationship between the structure of a protein encoded by a gene and its function.

Foreign genes or altered forms of an endogenous gene can be inserted into an organism. These techniques can result in the replacement of the endogenous gene, or in the integration of additional copies of it. Such introduced genes are called transgenes; the organisms carrying them are referred to as transgenics. Transgenes can be used to study organismal function and development in a variety of different ways. For instance, genes that are normally expressed at specific times and places during development can be genetically engineered in vitro to be expressed in different tissues at different times and then reintroduced into the animal to assess the cellular and organismal consequences.

The production of transgenic animals makes use of techniques for mutagenizing cloned genes in vitro and then transferring them into eukaryotic cells. Many types of cells can take up DNA from the medium. Yeast cells, for instance, can be treated with enzymes to remove their thick outer walls; the resulting spheroplasts will take up DNA added to the medium. Plant cells also can be converted to spheroplasts, which will take up DNA from the medium. Cultured mammalian cells take up DNA directly, particularly if it is first converted to a fine precipitate by treatment with calcium ions. Another popular method for introducing DNA into yeast, plant, and animal cells is called electroporation. Cells subjected to a brief electric shock of several thousand volts become transiently permeable to DNA. Presumably the shock briefly opens holes in the cell membrane allowing the DNA to enter the cells before the holes reseal. DNA also can be injected directly into the nuclei of both cultured cells and developing embryos.

Once the foreign DNA is inside the host cell, enzymes that probably function normally in DNA repair and recombination join the fragments of foreign DNA with the host cell's chromosomes. Since only a relatively small fraction of cells take up DNA, a selective technique must be available to identify the transgenic cells. In most cases the exogenous DNA includes a gene encoding a selectable marker such as drug resistance. The introduced DNA can insert into the host genome in a highly variable fashion showing no site specificity, can replace an endogenous gene by homologous recombination, or can remain as an independent extrachromosomal DNA molecule referred to as an episome.

Transgenic technology has numerous experimental applications and potential agricultural and therapeutic value. For instance, dominantly acting alleles of tumor-causing genes can be used to produce transgenic mice, thus providing an animal model for studying cancer. In Drosophila, transgenes often are used to determine whether a cloned segment of DNA corresponds to a gene defined by mutation. If the cloned DNA is indeed the gene in question, then introducing it as a transgene into a mutant fly will transform the mutant into a phenotypically normal individual. Transgenic plants may be commercially valuable in agriculture. Plant scientists, for example, have developed transgenic tomatoes that exhibit reduced production of ethylene, which promotes fruit ripening. The ripening process is delayed in these transgenic tomatoes, thus prolonging their shelf life. Finally, transgenic technology is a critical component in the burgeoning field of gene therapy for human genetic diseases.

The frequency of random integration of exogenous DNA into the mouse genome at nonhomologous sites is very high. Because of this phenomenon, the production of transgenic mice is a highly efficient and straightforward process.

The general process of making a transgenic mouse is as follows: foreign DNA containing a gene of interest is injected into one of the two pronuclei (the male and female haploid nuclei contributed by the parents) of a fertilized mouse egg before they fuse. The injected DNA has a good likelihood of being randomly integrated into the chromosomes of the diploid zygote. Injected eggs then are transferred to foster mothers in which normal cell growth and differentiation occurs. About 10-30 percent of the progeny will contain the foreign DNA in equal amounts (up to 100 copies per cell) in all tissues, including germ cells. Immediate breeding and backcrossing (parent-offspring mating) of the 10-20 percent of these mice that breed normally can produce pure transgenic strains homozygous for the transgene.

Numerous studies regarding the use of transgenic mice for studying various aspects of normal mammaliam biology have been published. These studies provide a model system for learning more about disease processes. For example, many forms of cancer are promoted by normal cellular genes acting in a dominant fashion owing to their misregulated activity. Although transgenic mice carrying one of these genes, called myc, develop normally, tumors form at a high frequency. The observation that only a small number of cells expressing the transgene develop tumors supports a model in which additional genetic changes are necessary for tumors to form. These mice may provide an important tool for identifying those changes.

Cleavage Mechanisms of Ribozymes

A general description of ribozymes is found in Fedor, M. J. and Westhof, E., Mol. Cell., 10(4):703-704 (2002), and the mechanisms of action of various types of ribozymes are discussed in Takagi, Y., et al., Nucleic Acids Res., 29(9):1815-1834 (2001).

Group I introns were originally identified as an intervening sequence and defined based on conserved sequences and secondary structure elements. Group I introns are widely distributed, nearly 1000 group I introns have been found in the nuclear, mitochondrial, and chloroplast genomes of eukaryotes, in eubacteria, and in bacteriophages. Group II introns were also identified as an intervening sequence and defined based on conserved sequences and secondary structure elements. Group II introns are also widely distributed, about 100 group II introns have been found in the rRNA, tRNA, and mRNA of organelles in fungi, protists, and plants, and in the mRNA of bacteria (Bonen, L., and Vogel, J., TRENDS Genet., 17:322 (2001)).

Group I introns fold to form an active site to mediate their own RNA splicing. Sequence elements conserved among an available set of 66 group I introns were compiled. Comparative sequence analysis led to the prediction of some conserved structural features. The significance of these conserved features is discussed in Cech, T. R., Gene, 73(2):259-271 (1988). In addition, a review on the self-splicing nature of group I introns is presented in Cech, T. R., Annu. Rev. Biochem., 59:543-568 (1990).

Group II introns are found in eubacteria and eubacteria-derived organellar genomes. They have ribozymic activities by which they direct and catalyze the splicing of the exons flanking them. The secondary structure and known tertiary interactions of the ribozymic component of group II introns is discussed in Michel, F. and Ferat, J. L., Annu. Rev. Biochem., 64:435-461 (1995).

In the case of group I and II intronic ribozymes, possible cleavage mechanisms include, but are not limited to, self-cleavage or splicing via transesterification, and hydrolysis. Such reactions may be driven by a simple acid-base reaction, or by nucleophilic substitution. Group II introns are discussed in Bonen, L. and Vogel, J., Trends. Genet., 17(6):322-331 (2001).

Both of the hammerhead and hairpin ribozymes can be engineered to cleave any target RNA that contains a GUC sequence (Haseloff et al., Nature, 334, 585-591 (1988); Uhlenbeck, Nature, 334, 585 (1987); Hampel et al., Nuc. Acids Res., 18, 299-304 (1990); and Symons, Ann. Rev. Biochem., 61, 641-671 (1992)). Generally speaking, hammerhead ribozymes have two types of functional domains, a conserved catalytic domain flanked by two hybridization domains. The hybridization domains bind to sequences surrounding the GUC sequence and the catalytic domain cleaves the RNA target 3′ to the GUC sequence (Uhlenbeck (1987), supra; Haseloff et al. (1988), supra; and Symons (1992), supra).

Additional information concerning the structural bases of hammerhead ribozyme self-cleavage can be found in Murray, J. B., et al., Cell, 92(5):665-673 (1998). Further information regarding the structure and function of the hairpin ribozyme and the catalytic mechanism of the hairpin ribozyme can be found in Fedor, M. J., J. Mol. Biol. 297(2):269-291 (2000), and Fedor, M. J., Biochem. Soc. Trans., 30:1109-1115 (2002).

One ribozyme that can be used in the transcription unit of the invention is a hammerhead ribozyme that is derived from the satellite RNA of tobacco ringspot virus (sTobRV). sTobRV undergoes self-catalyzed cleavage during replication. The sequences required for (+) and (−) strand cleavage have been determined (Haseloff, J. and Gerlach, W. L., Gene, 82:43-52 (1989)). Cleavage of the (+) strand requires those sequences flanking the site for cleavage to form a “hammerhead” domain, similar to those found in other satellite and viriod RNA. Specifically, cleavage of the (+) strand occurs after the sequence GUC and results in the production of termini containing 5′ hydroxyl and 2′, 3′ cyclic phosphodiester groups (Prody, G. A., et al., Science, 231:1577-1580 (1986)). A well recognized secondary-structure motif underlies cleavage of sTobRV (+) strands, and it is likely that this highly conserved structure is directly involved in catalysis. Cleavage of the (−) strand requires only a small region of 12 nucleotides at the site of cleavage, and a sequence of 55 nucleotides positioned elsewhere in the molecule. The RNA structure which is associated with cleavage of the sTobRV (−) strand may similarly play a role in catalysis and comprise a novel structural motif which will be found reiterated in other catalytic RNAs.

In addition, a sequence of a 300 nucleotide satellite RNA associated with the Arabis mosaic virus (ArMv) has also been reported (Kaper, J. M., et al., Biochem. Biophys. Res. Commun. 154:318-325 (1988). A ribozyme derived from the satellite RNA associated with ArMV can also be used in the transcription unit of the invention. ArMV is a nepovirus related to TobRV, and its satellite RNA. sArMV shares 50% sequence similarity with sTobRv. The presence of conserved sequences and potential base-pairing was used to identify the domain likely to be associated with sArMV (+) strand cleavage. The structure of the domain and the nucleotides involved in cleavage are discussed by Kaper, J. M., et al. described above. Conserved regions exist between sTobRV, sArMV, and other satellite and viriod RNA, as well as similar secondary structures. Accordingly, ribozymes derived from other satellite and viriod RNA can also be used in the transcription unit of the invention.

Cleavage Signals

Cleavage signals that can be inserted into the non-coding region of the transcription unit can be, for example, a polyadenylation signal, a pause site, a strong pause site, a termination site, a near upstream (NUE), or a 3′ untranslated sequence.

It is known that following transcript initiation RNA polymerase can pause at several locations called transient pause sites, pause sites, strong pause sites, and termination sites. This phenomenon is described, for example, in Landick, R., Cell, 88:741-744 (1997), and Reeder, R. H. and Lang, W., Mol. Microbiol., 12:11-15 (1994).

Regulatory Sequences and Coding Sequences

Useful regulatory sequences can comprise for example, a viral long terminal repeat (LTR), such as the LTR of the Moloney murine leukemia virus, the early and late promoters of SV40, adenovirus or cytomegalovirus immediate early promoter, the lac system, the trp system, the TAC or TRC system, the T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast alpha-mating factors, the polyhedron promoter of the baculovirus system, and other sequences known to control the expression of genes of prokaryotic or eukaryotic cells or their viruses, and various combinations thereof. Suitable eukaryotic promoters include the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

The constructs of the invention, especially the regulatory and coding sequence portions thereof, may comprise sequences that are viral in origin. Thus, viral regulatory sequences or regions (which act in cis) and coding regions (which act in trans) can be used in the practice of the invention. Examples of cis acting regions are the TAR and RRE, INS (inhibitory sequence or instability sequence, also referred to as CRS) elements of retroviruses, while examples of trans acting coding regions are the tat and rev coding sequences.

For example, a RRE heterologous to the viral nucleic acid of interest can be used in the vectors of the invention. Examples of suitable RREs include, but are not limited to, HIV-2 RRE for an HIV-1 derived nucleic acid, a CTE (constitutive transport element such as that from Mason-Pfizer monkey virus and other retroviruses) or a PRE (post-transcriptional regulatory element such as that from the woodchuck hepatitis virus). RREs are RNA sequences that control transport of the RNA from the nucleus to the cytoplasm for translation.

Selection of appropriate vectors and promoters for propagation or expression in a host cell is a well known procedure. And the requisite techniques for vector construction, introduction of the vector into the host, and propagation or expression in the host are routine to those skilled in the art. It will be understood that numerous promoters and other control sequences not mentioned above are suitable for use in this aspect of the invention, are well known, and may be readily employed by those of skill in the art.

Vectors

Vectors that can be used in the present invention are described below. As used herein, the term “vector” refers to a nucleic acid molecule capable of transporting another nucleic acid to which it has been linked. One type of vector is an episome, i.e., a nucleic acid capable of extra-chromosomal replication. Other vectors are capable of autonomous replication and/expression of nucleic acids to which they are linked. Vectors capable of directing the expression of genes to which they are operably linked are referred to herein as “expression vectors.” In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably. In addition, the invention is intended to include other forms of vectors which serve equivalent functions and which become known in the art subsequently hereto.

Vectors can be used for the expression of polynucleotides and polypeptides. Generally, such vectors comprise cis-acting control regions effective for expression in a host operably linked to the polynucleotide to be expressed. Appropriate trans-acting factors either are supplied by the host, supplied by a complementing vector, or supplied by the vector itself upon introduction into the host.

A great variety of vectors can be used in the invention. Such vectors include chromosomal, episomal, virus-derived vectors, vectors derived from bacterial plasmids, from bacteriophage, from yeast episomes, from yeast chromosomal elements, from viruses such as baculoviruses, papovaviruses, such as SV40, vaccinia viruses, adenoviruses, fowl pox viruses, pseudo-rabies viruses and retroviruses, and vectors derived from combinations thereof, such as those derived from plasmid and bacteriophage genetic elements, such as cosmids and phagemids. Generally, any vector suitable to maintain, propagate or express polynucleotides in a host may be used.

The following vectors, which are commercially available, are provided by way of example. Among vectors for use in bacteria are pQE70, pQE60, and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Eukaryotic vectors available are pWLNEO, pSV2CAT, pOG44, pXT1, and pSG available from Stratagene; and pSVK3, pBPV, pMSG, and pSVL available from Pharmacia. These vectors are listed solely by way of illustration of the many commercially available and well known vectors that are available to those of skill in the art for use in accordance with the present invention. It will be appreciated that any other plasmid or vector suitable for, for example, introduction, maintenance, propagation, and/or expression of a polynucleotide or polypeptide of the invention in a host may be used in this aspect of the invention.

The appropriate DNA sequence may be inserted into the vector by any of a variety of well-known and routine techniques. In general, a DNA sequence is joined to a vector by cleaving the DNA sequence and the vector with one or more restriction endonucleases and then joining the restriction fragments together using a DNA ligase. Procedures for restriction and ligation that can be used are well known and routine to those of skill in the art. Suitable procedures in this regard, and for constructing vectors using alternative techniques, which also are well known and routine to those skilled in the art, are set forth in great detail in Sambrook et al.cited elsewhere herein.

The sequence in the vector is operably linked to appropriate expression control sequence(s), including, for instance, a promoter to direct mRNA transcription.

It should be understood that the choice and/or design of the vector may depend on such factors as the choice of the host cell to be transformed and/or the type of protein(s) desired to be expressed. Moreover, the vector's copy number, the ability to control that copy number, and the expression of any other proteins encoded by the vector, such as antibiotic markers, should also be considered. Expression vectors can be used to transfect cells and thereby replicate regulatory sequences and produce proteins or peptides, including those encoded by nucleic acids as described herein.

Genetic Engineering of Cells

The transcriptional units of the invention may be incorporated into vectors and/or introduced into cells, such as, but not limited to, mammalian, rodent, primate, or human cells. The constructs of the invention may be integrated into the cellular genome or maintained as episomal constructs. The constructs of the invention may be introduced into cells in any order. After introduction, the presence of the constructs in said cells may be confirmed by detecting said constructs via a selectable or detectable marker placed on said construct.

Host cells can be genetically engineered to incorporate polynucleotides and express polypeptides of the present invention. For instance, polynucleotides may be introduced into host cells using well known techniques of infection, transduction, transfection (for example, electroporation, lipofection, and calcium phosphate precipitation), transvection, and transformation. The polynucleotides may be introduced alone or with other polynucleotides. Such other polynucleotides may be introduced independently, co-introduced, or introduced joined to the polynucleotides of the invention.

Thus, for instance, polynucleotides of the invention may be transfected into host cells with another, separate, polynucleotide encoding a selectable marker, using standard techniques for co-transfection and selection in, for instance, mammalian cells. In this case the polynucleotides generally will be stably incorporated into the host cell genome.

In addition, the polynucleotides may be joined to a vector containing a selectable marker for propagation in a host. The vector construct may be introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it may be packaged in vitro or introduced into a packaging cell and the packaged virus may be transduced into cells. A wide variety of techniques suitable for making polynucleotides and for introducing polynucleotides into cells in accordance with this aspect of the invention are well known and routine to those of skill in the art. Such techniques are reviewed at length in Sambrook et al., which is illustrative of the many laboratory manuals that detail these techniques. In addition, the vector may be, for example, a plasmid vector, a single or double-stranded phage vector, a single or double-stranded RNA or DNA viral vector. Such vectors may be introduced into cells as polynucleotides, such as DNA, by well known techniques for introducing DNA and RNA into cells. The vectors, in the case of phage and viral vectors may be introduced into cells as packaged or encapsidated virus by well known techniques for infection and transduction. Viral vectors may be replication competent or replication defective. In the latter case viral propagation generally will occur only in complementing host cells.

As used herein, the term “transfection” means the introduction of a nucleic acid, e.g., an expression vector, into a recipient cell by nucleic acid-mediated gene transfer. “Transformation,” as used herein, refers to a process in which a cell's genotype is changed as a result of the cellular uptake of exogenous DNA or RNA. For example, a transformed cell expresses a recombinant form of a polypeptide or, where anti-sense expression occurs from the transferred gene, the expression of a naturally-occurring form of a protein is disrupted.

Transfection can be either transient transfection or stable transfection. Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis, et al., Basic Methods In Molecular Biology (1986).

Cell or Host

As used herein, a “cell” or “host” refers to the corresponding living organism in which the nucleic acid constructs or expression systems of the invention may be introduced and expressed. A “cell” may be any cell, and, preferably, is a eukaryotic cell. The cells may be those of a cell line or primary cells newly isolated and transformed by, or in conjunction with, the introduction of the nucleic acid constructs of the invention. Cell lines or cultures refer to cells maintained via in vitro culturing which may be non-identical to the parental cell(s) from which the lines or cultures were derived. Non-limiting examples of cells include eukaryotic cell lines, such as HeLa, 293, HT-1080, CV-1, TE671 or other human cells; Vero cells; or D17 cells. Other cells include a lymphocyte (such as T or B cells) or a macrophage (such as a monocytic macrophage), or is a precursor to either of these cells, such as a hematopoietic stem cell. Additional cells for the practice of the invention include an astrocyte, a skin fibroblast, a bowel epithelial cell, an endothelial cell, an epithelial cell, a dendritic cell, Langerhan's cells, a monocyte, a muscle cell, a neuronal cell (such as, but not limited to brain and eye), a hepatocyte, a hematopoietic stem cell, an embryonic stem cell, a cell that give rise to spermatozoa or an oocyte, a stromal cell, a mucosal cell and the like. Preferably, the host cell is of a eukaryotic, multicellular species (e.g., as opposed to a unicellular yeast cell), and, even more preferably, is a mammalian, e.g., human, cell.

A cell can be present as a single entity, or can be part of a larger collection of cells. Such a “larger collection of cells” can comprise, for instance, a cell culture (either mixed or pure), a tissue (e.g., endothelial, epithelial, mucosa or other tissue, including tissues containing the above mentioned CD 4 lacking cells), an organ (e.g., heart, lung, liver, muscle, gallbladder, urinary bladder, gonads, eye, and other organs), an organ system (e.g., circulatory system, respiratory system, gastrointestinal system, urinary system, nervous system, integumentary system or other organ system), or an organism (e.g., a bird, mammal, or the like). Preferably, the organs/tissues/cells are of the circulatory system (e.g., including, but not limited to heart, blood vessels, and blood, including white blood cells and red blood cells), respiratory system (e.g., nose, pharynx, larynx, trachea, bronchi, bronchioles, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including, but not limited to, brain and spinal cord, and special sense organs, such as the eye) and integumentary system (e.g., skin, epidermis, and cells of subcutaneous or dermal tissue). Even more preferably, the cells are selected from the group consisting of heart, blood vessel, lung, liver, gallbladder, urinary bladder, and eye cells. The cells need not be normal cells and can be diseased cells. Such diseases cells can be, but are not limited to, tumor cells, infected cells, genetically abnormal cells, or cells in proximity or contact to abnormal tissue such as tumor vascular endothelial cells.

Virus

A “virus” is an infectious agent that consists of protein and nucleic acid, and that uses a host cell's genetic machinery to produce viral products specified by the viral nucleic acid. The invention includes aspects, such as expression of viral coding sequences, that may be applied to both RNA and DNA viruses. RNA viruses are a diverse group that infects prokaryotes (e.g., the bacteriophages) as well as many eukaryotes, including mammals and, particularly, humans. Most RNA viruses have single-stranded RNA as their genetic material, although at least one family has double-stranded RNA as the genetic material. The RNA viruses are divided into three main groups: the positive-stranded viruses, the negative-stranded viruses, and the double-stranded RNA viruses. RNA viruses related to the present invention includes Sindbis-like viruses (e.g., Togaviridae, Bromovirus, Cucumovirus, Tobamovirus, Ilarvirus, Tobravirus, and Potexvirus), Picornavirus-like viruses (e.g., Picornaviridae, Caliciviridae, Comovirus, Nepovirus, and Potyvirus), minus-stranded viruses (e.g., Paramyxoviridae, Rhabdoviridae, Orthomyxoviridae, Bunyaviridae, and Arenaviridae), double-stranded viruses (e.g., Reoviridae and Birnaviridae), Flavivirus-like viruses (e.g., Flaviviridae and Pestivirus), Retrovirus-like viruses (e.g., Retroviridae), Coronaviridae, and other viral groups including, but not limited to, Nodaviridae. The invention is applied preferably to an RNA virus of the family Flaviviridae, more preferably a virus of the genus Filovirus, and especially a Marburg or Ebola virus. A virus of the family Flaviviridae is a virus of the genus Flavivirus, such as yellow fever virus, dengue virus, West Nile virus, St. Louis encephalitis virus, Japanese encephalitis virus, Murray Valley encephalitis virus, Rocio virus, tick-borne encephalitis virus, and the like. The invention is preferably applied to a virus of the family Picornaviridae, preferably a hepatitis A virus (HAV), hepatitis B virus (HBV), hepatitis C virus (HBC), or a non-A or non-B hepatitis virus.

Another preferred RNA virus to which the invention may be applied is a virus of the family Retroviridae (i.e., a retrovirus), particularly a virus of the genus or subfamily Oncovirinae, Spumavirinae, Spumavirus, Lentivirinae, and Lentivirus. An RNA virus of the subfamily Oncovirinae is desirably a human T-lymphotropic virus type 1 or 2 (i.e., HTLV-1 or HTLV-2) or bovine leukemia virus (BLV), an avian leukosis-sarcoma virus (e.g., Rous sarcoma virus (RSV), avian myeloblastosis virus (AMV), avian erythroblastosis virus (AEV), and Rous-associated virus (RAV; RAV-0 to RAV-50), a mammalian C-type virus (e.g., Moloney murine leukemia virus (MuLV), Harvey murine sarcoma virus (HaMSV), Abelson murine leukemia virus (A-MuLV), AKR-MuLV, feline leukemia virus (FeLV), simian sarcoma virus, reticuloendotheliosis virus (REV), spleen necrosis virus (SNV)), a B-type virus (e.g., mouse mammary tumor virus (MMTV)), and a D-type virus (e.g., Mason-Pfizer monkey virus (MPMV) and “SAIDS” viruses). An RNA virus of the subfamily Lentivirus is desirably a human immunodeficiency virus type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. The acronym “HIV” or “human immunodeficiency virus” are used herein to refer to these HIV viruses, and HIV-related and -associated viruses, generically. Moreover, an RNA virus of the subfamily Lentivirus preferably is a Visna/maedi virus (e.g., such as infect sheep), a feline immunodeficiency virus (FIV), bovine lentivirus, simian immunodeficiency virus (SIV), an equine infectious anemia virus (EIAV), and a caprine arthritis-encephalitis virus (CAEV). The invention may also be applied to a DNA virus. Preferably, the DNA virus is an herpes virus (such as Epstein-Barr virus, herpes simplex viruses, cytomegalovirus) an adenovirus, an AAV, a papilloma virus, a vaccinia virus, and the like.

3′

The term “3′” (three prime) generally refers to a region or position in a polynucleotide or oligonucleotide 3′ (downstream) from another region or position in the same polynucleotide or oligonucleotide.

5′

The term “5′” (five prime) generally refers to a region or position in a polynucleotide or oligonucleotide 5′ (upstream) from another region or position in the same polynucleotide or oligonucleotide.

Unless defined otherwise all technical and scientific terms used herein have the same meaning as commonly understood to one of ordinary skill in the art to which this invention belongs.

It must be noted that as used in this specification and the appended claims, the singular forms “a”, “an” and “the” include corresponding plural references unless the context clearly dictates otherwise.

As used herein, the term “comprising” and its cognates are used in their inclusive sense; that is, equivalent to the term “including” and its corresponding cognates.

The practice of the present invention will employ, unless otherwise indicated, conventional techniques of cell biology, cell culture, molecular biology, microbiology, recombinant, which are within the skill of one skilled in the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195: Nucleic Acid Hybridization (B. D. Hames & S. J. Higgins eds. 1984); Transcription And Translation (B. D. Hames & S. J. Higgins eds. 1984); Culture Of Animal Cells (R. I. Freshney. Alan R. Liss, Inc., 1987); Immobilized Cell And Enzymes (IRL, Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al.eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLE 1

### Structure of the cis-Acting Ribozyme in a Packaging Vector Construct VRX577

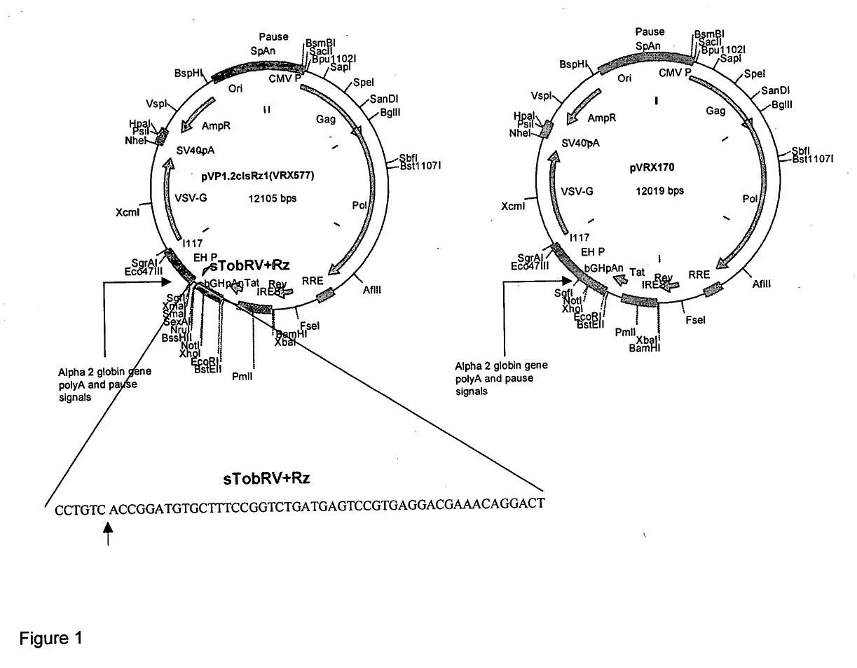

In the packaging vector construct VIRPAC, also known as VRX170, there are two transcriptional units. One drives the expression of the HIV-1 GagPol and TatRev under the control of the cytomegalovirus (CMV) promoter, which terminates at the end of the bovine growth hormone polyadenylation (poly-A) signal. The other transcriptional unit encodes VSV-G driven by the elongation factor (EF) promoter and terminates at the end of the SV40 poly-A site. Because of the circular nature of the plasmid, either transcriptional unit could potentially read-through the poly A site and result in a longer polycistronic messenger RNA which could serve as a potential substrate for generation of replication competent lentiviruses (RCL) through RNA-based recombination events during reverse transcription. To prevent read-through, a fragment containing the cis ribozyme (cis-Rz) of the satellite RNA of tobacco ringspot virus (sTobRV) (Haseloff and Gerlach, 1989) was inserted between these two transcriptional units immediately downstream of corresponding poly-A sites (FIG. 1). Only in the event of transcriptional read-through will be ribozyme be made, which was then able to cleave itself at the site indicated by the black arrow in FIG. 1.

### EXAMPLE 2

### In Vitro Activity of the cis-Rz

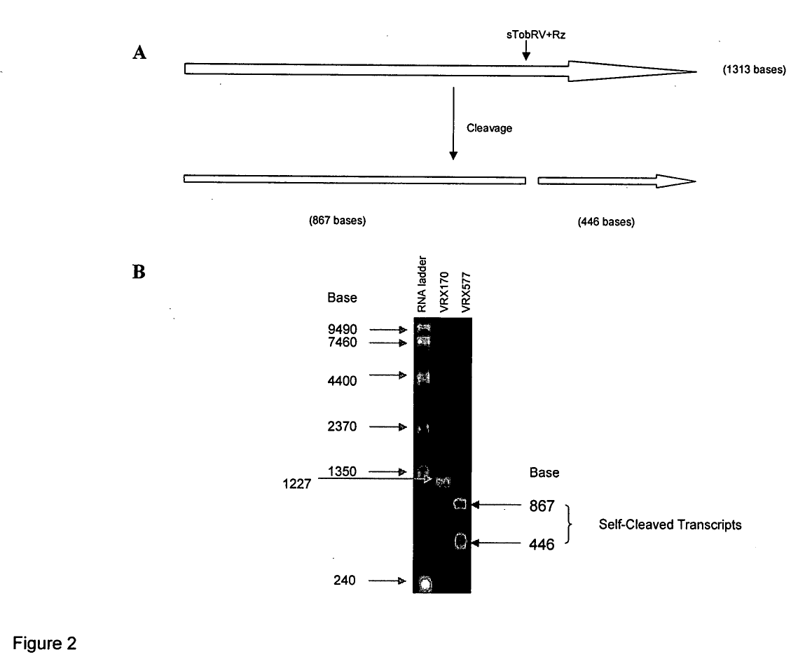

A 1300-base region of VRX170 (− cis-RZ) and VRX577 (+ cis-Rz) containing the transcriptional stop elements was amplified by PCR using primers containing a T7 promoter. The resulting DNA was then transcribed in vitro. 2 μl of transcribed RNA at a concentration of about 1 μg/μl was added to 2 μl of RT-PCR buffer, then 2 μl (2 μg) was loaded onto the gel for visualization (FIG. 2). Cleavage occurred rapidly, as no difference between 5, 10, 20, and 60 minutes of incubation in buffer prior to gel loading was observed. This data indicates that the cis-Rz very efficiently inhibits read-through transcripts.

### EXAMPLE 3

### Lack of Transcriptional Read-Through in Production Cells Using VRX577 as a Packaging Vector

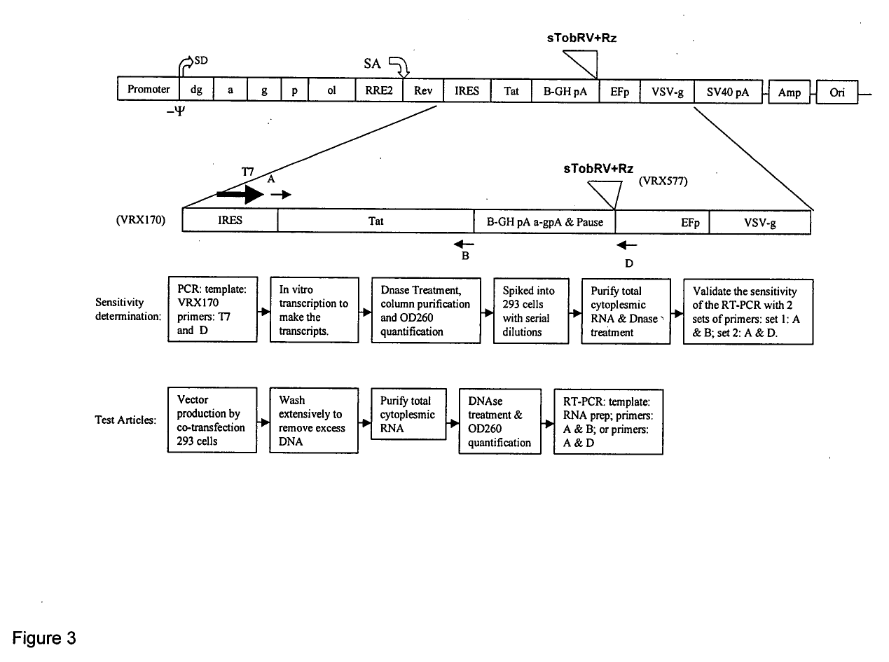

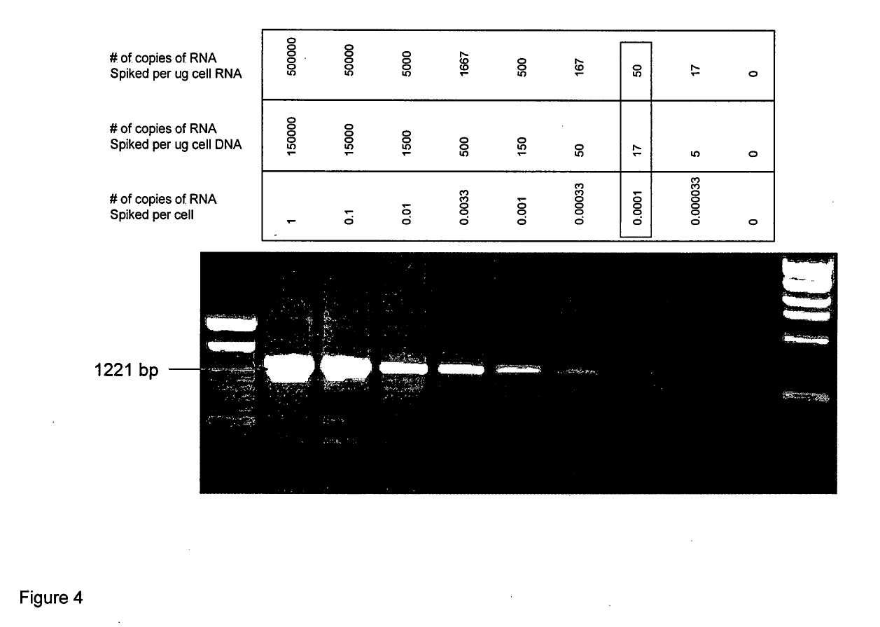

Transcriptional read-through was examined in 293F cells cotransfected with a viral vector and a helper construct containing the cis-Rz (VRX577) and one without the cis-Rz (VRX170). Cellular RNA was isolated and RNA transcripts were analyzed by RT-PCR. The assay sensitivity at which transcripts can be detected 100% of the time is 50 copies of transcript per μg of cellular DNA, which is equal to 167 copies per μg of total cellular RNA. An overview of the PCR primer design in addition to a summary of the experimental design for detection of transcriptional read-through is presented in FIG. 3. In vitro transcribed RNA from VRX170 was used as the positive control and standard. Read-through was expected during in vitro transcription with VRX170, since the cellular elements necessary to engage the transcriptional poly-A and pause sites are not present in the reaction. To statistically determine the limit of detection of the assay, known amounts of positive control RNA were diluted in a 3-fold dilution series (FIG. 4), and the experiment was repeated 13 times to achieve sufficient data points for. statistical determination of the sensitivity. According to these results, if there are no positive events in 9 replicates, there are fewer than 23.12 copies of read-thorugh RNA transcript present per replicate, or μg of RNA, at the 95% upper confidence limit.

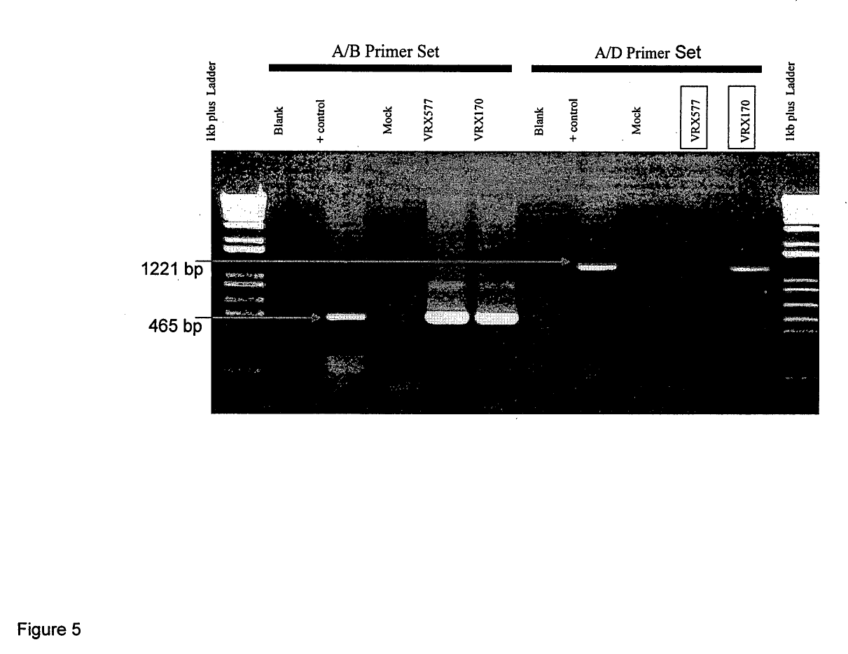

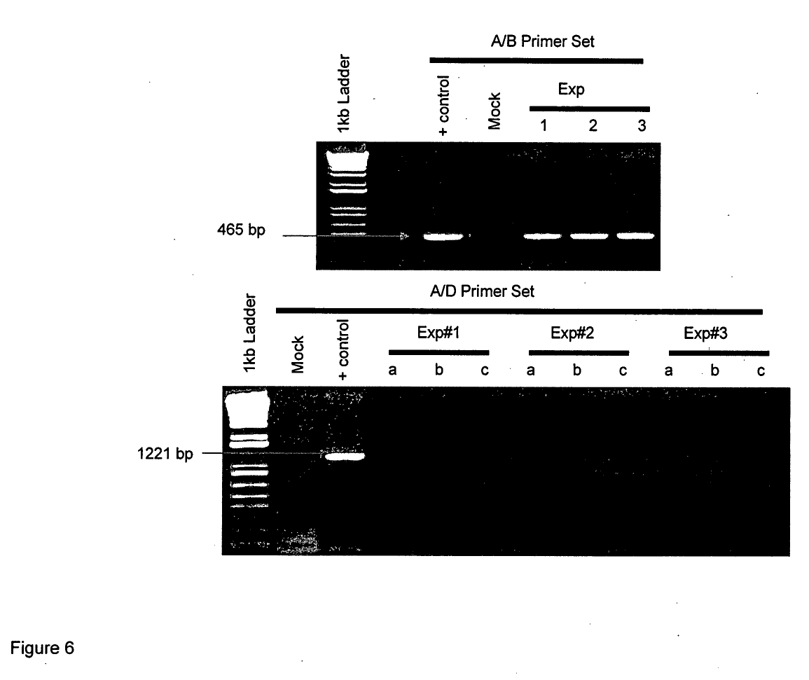

Read-through transcripts were detected in 293F cells using VRX170 as the helper construct, but none were detected with VRX577 as the helper construct at the assay sensitivity described (FIG. 5). There were no read-through transcripts in any of 9 subsequent assays performed on cellular RNA from cells cotransfected with vector and VRX577, which means that there are fewer than 23.12 copies, or read-through transcripts, per μg of cellular RNA (FIG. 6). Since the only difference between VRX170 and VRX577 is in the addition of the cis-acting ribozyme (please refer to FIG. 1), it can be concluded that the ribozyme is solely responsible for preventing read-through transcripts. Therefore, the addition of a cis-acting ribozyme is an extremely effective transcriptional separating element.

### EXAMPLE 4

### Addition of a cis-Rz Does Not Affect the Packaging Function of the Helper Construct

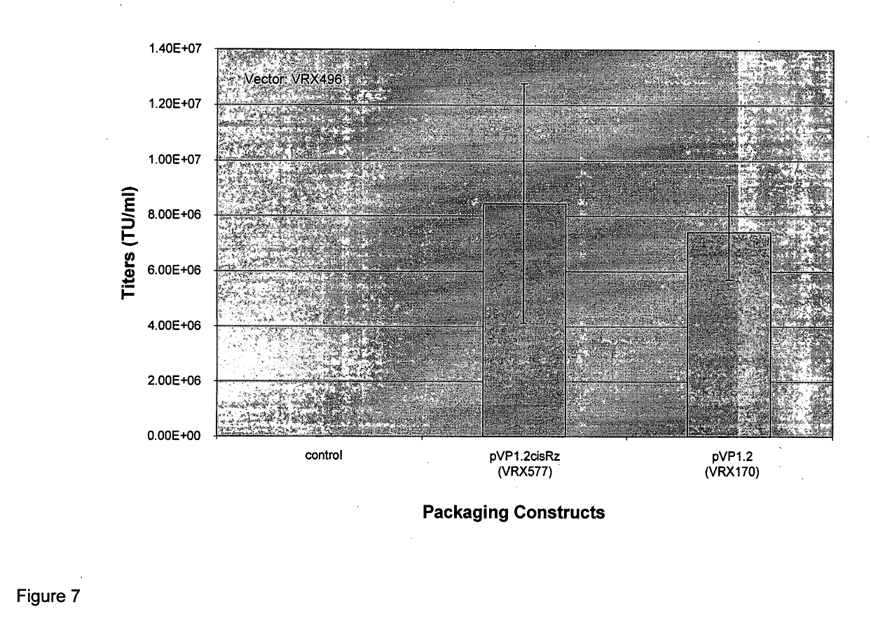

The use of the cis-Rz does not affect the final viral vector product in any way during packaging, since the ribozyme is not located in the viral vector or in a coding sequence in the helper (VRX577). To demonstrate this, the resulting titers of vector packaged in the presence of either VRX170 or VRX577 were compared. Briefly, 293F cells were cotransfected with vector and helper constructs, and the resultant vector product was used to transduce hela-tat cells. DNA was isolated from these cells, and assayed for vector copy number (transduction units (TU)) by PCR amplification of the vector sequence. No difference between the efficacy of vector packaging was observed. The data is representative of at least 3 separate experiments which demonstrated comparable titers between the two helpers (VRX577 and VRX170), with a reproducible trend towards higher titers when using VRX577 (FIG. 7).

All references cited herein, including patents, patent applications, and publications, are hereby incorporated by reference in their entireties, whether previously specifically incorporated or not.

Citation of the above documents is not intended as an admission that the foregoing are pertinent prior art. All statements as to the dates or representation as to the contents of these documents is based on the information available to the applicant and does not constitute any admission as to the correctness of the dates or contents of these documents.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of preparing a recombinant transcription unit capable of producing an RNA transcript of a predetermined size comprising:
operably linking a regulatory sequence and a nucleotide sequence comprising a transcribed region such that transcription of said transcribed region is controlled by said regulatory sequence, wherein said transcribed region comprises a region that encodes a viral sequence and a non-coding region downstream of said region encoding for said viral sequence, wherein said non-coding region comprises a nucleotide sequence encoding a cis-acting ribozyme.

2. The method of claim 1, wherein said non-coding region further comprises a nucleotide sequence encoding a cleavage signal upstream of said nucleotide sequence encoding a cis-acting ribozyme.

3. The method of claim 2, wherein said cleavage signal is a polyadenylation signal, a transient pause site, a strong pause site, a termination site, a near upstream (NUE), or a 3′ untranslated sequence.

4. The method of claim 3, wherein said polyadenylation signal is a bovine growth hormone polyadenylation (poly-A) signal or a S4V0 poly-A site.

5. The method of claim 3, wherein more than one cleavage signal is used.

6. The method of claim 1, wherein said regulatory sequence is a prokaryotic regulatory sequence.

7. The method of claim 1, wherein said regulatory sequence is a eukaryotic regulatory sequence.

8. The method of claim 7, wherein said regulatory sequence is a cytomegalovirus (CMV) promoter or an elongation factor (EF) promoter.

9. The method of claim 1, wherein said viral sequence encodes a viral protein.

10. The method of claim 9, wherein said viral protein is a protein encoded by a lentivirus or a viral envelope protein.

11. The method of claim 9, wherein said viral protein is VSV-G, gag, pol, tat, or rev, or any combination of VSV-G, gag, pol, tat, and rev.

12. The method of claim 9, wherein said viral sequence further comprises a nucleotide sequence encoding an antiviral agent that is either upstream or downstream of the nucleotide sequence encoding said viral protein.

13. The method of claim 12, wherein said antiviral agent is an antisense molecule or a ribozyme.

14. The method of claim 1, wherein said cis-acting ribozyme is derived from satellite or viroid RNA.

15. The method of claim 14, wherein said cis-acting ribozyme is derived from satellite RNA of Tobacco Ringspot Virus or derived from satellite RNA of Arabis mosaic virus.

16. A host cell comprising a recombinant transcription unit capable of producing an RNA transcript of a predetermined size, wherein said transcription unit comprises a regulatory sequence operably linked to a nucleotide sequence comprising a transcribed region such that the transcription of said transcribed region is controlled by said regulatory sequence, wherein said transcribed region comprises a region that encodes for a viral sequence, and a non-coding region downstream of said region encoding for said viral sequence, wherein said non-coding region comprises a nucleotide sequence encoding a cis-acting ribozyme.

17. The host cell of claim 16, wherein said non-coding region further comprises a nucleotide sequence encoding a cleavage signal upstream of said nucleotide sequence encoding said cis-acting ribozyme.

18. A recombinant transcription unit capable of producing an RNA transcript of a predetermined size comprising a regulatory sequence operably linked to a nucleotide sequence comprising a transcribed region encoding a viral sequence and a non-coding region downstream of said region encoding for said viral sequence, wherein said non-coding region comprises a nucleotide sequence encoding a cis-acting ribozyme.

19. The recombinant transcription unit of claim 18, wherein said non-coding region further comprises a nucleotide sequence encoding a termination cleavage signal upstream of said nucleotide sequence encoding said cis-acting ribozyme.

20. The recombinant transcription unit of claim 19, wherein said cleavage signal is a polyadenylation signal, a pause site, a strong pause site, a near upstream (NUE), or a 3′ untranslated sequence.

21. The recombinant transcription unit of claim 20, wherein said polyadenylation signal is a bovine growth hormone polyadenylation (poly-A) signal, or a SV40 poly-A site.

22. The recombinant transcription unit of claim 20, wherein more than one signal is used.

23. The recombinant transcription unit of claim 18, wherein said regulatory sequence is a prokaryotic regulatory sequence.

24. The recombinant transcription unit of claim 18, wherein said regulatory sequence is a eukaryotic regulatory sequence.

25. The recombinant transcription unit of claim 24, wherein said regulatory sequence is a cytomegalovirus (CMV) promoter or an elongation factor (EF) promoter.

26. The recombinant transcription unit of claim 18, wherein said viral sequence is a viral protein.

27. The recombinant transcription unit of claim 26, wherein said viral protein is a protein encoded by a lentivirus or a viral envelope protein.

28. The recombinant transcription unit of claim 26, wherein said viral protein is VSV-G, gag, pol, tat, or rev, or any combination of VSV-G, gag, pol, tat, and rev.

29. The recombinant transcription unit of claim 28, wherein in addition to a nucleotide sequence encoding a viral protein said viral sequence further comprises a nucleotide sequence encoding an antiviral agent that is either upstream or downstream of the nucleotide sequence encoding said viral protein.

30. The recombinant transcription unit of claim 29, wherein said antiviral agent is an antisense molecule or a ribozyme.

31. The recombinant transcription unit of claim 18, wherein said cis-acting ribozyme is derived from satellite or viroid RNA.

32. The recombinant transcription unit of claim 31, wherein said cis-acting ribozyme is derived from satellite RNA of Tobacco Ringspot Virus or derived from satellite RNA of Arabis mosaic virus.

33. A method of limiting the size of an RNA transcript produced from a transcription unit, said method comprising:
inducing transcription of a transcription unit comprising a regulatory sequence operably linked to a nucleotide sequence comprising a transcribed region such that the transcription of said transcribed region is controlled by said regulatory sequence, wherein said transcribed region comprises a region that encodes for a viral sequence, and a non-coding region downstream of said region encoding for said viral sequence, wherein said non-coding region comprises a nucleotide sequence encoding a cis-acting ribozyme; and
wherein said transcription unit produces a transcript under conditions wherein the sequence encoding said cis-acting ribozyme is transcribed and cleaves said transcript in cis.

34. The method of claim 33, wherein said non-coding region further comprises a nucleotide sequence encoding a cleavage signal upstream of said nucleotide sequence encoding a cis-acting ribozyme.

35. The method of claim 33, wherein said cleavage signal is a polyadenylation signal, a transient pause site, a strong pause site, a termination site, a near upstream (NUE), or a 3′ untranslated sequence.

36. The method of claim 33, wherein said polyadenylation signal is a bovine growth hormone polyadenylation (poly-A) signal, or a S4V0 poly-A site.

37. The method of claim 35, wherein more than one signal is used.

38. The method of claim 33, wherein said regulatory sequence is a prokaryotic regulatory sequence.

39. The method of claim 33, wherein said regulatory sequence is a eukaryotic regulatory sequence.

40. The method of claim 39, wherein said regulatory sequence is a cytomegalovirus (CMV) promoter or an elongation factor (EF) promoter.

41. The method of claim 33, wherein said viral sequence encodes a viral protein.

42. The method of claim 41, wherein said viral protein is a protein encoded by a lentivirus or a viral envelope protein.

43. The method of claim 41, wherein said viral protein is VSV-G, gag, pol, tat, or rev, or any combination of VSV-G, gag, pol, tat, and rev.

44. The method of claim 41, wherein in addition to a nucleotide sequence encoding a viral protein said viral sequence further comprises a nucleotide sequence encoding an antiviral agent that is either upstream or downstream of the nucleotide sequence encoding said viral protein.

45. The method of claim 44, wherein said antiviral agent is an antisense molecule or a ribozyme.

46. The method of claim 33, wherein said cis-acting ribozyme is derived from satellite or viroid RNA.

47. The method of claim 46, wherein said cis-acting ribozyme is derived from satellite RNA of Tobacco Ringspot Virus or derived from satellite RNA of Arabis mosaic virus.

48. A vector comprising:
(a) a first transcription unit capable of producing a first RNA transcript of a predetermined size, wherein said first transcription unit comprises a first promoter operably linked to a nucleotide sequence comprising a transcribed region such that the transcription of said transcribed region is controlled by said first promoter, wherein said transcribed region comprises a region that encodes for a first gene, and a first non-coding region downstream of said region encoding for said first gene, wherein said first non-coding region comprises a nucleotide sequence encoding a cis-acting ribozyme; and
(b) a second transcription unit capable of producing a second RNA transcript of a predetermined size, wherein said second transcription unit comprises a second promoter operably linked to a nucleotide sequence comprising a transcribed region such that the transcription of said transcribed region is controlled by said second promoter, wherein said transcribed region comprises a region that encodes for a second gene, and a second non-coding region downstream of said region encoding for said second gene, wherein said second non-coding region comprises a nucleotide sequence encoding a cis-acting ribozyme.

49. The vector of claim 48, wherein said first and second promoter are different.

50. The vector of claim 48, wherein said first and second promoter non-coding regions comprise a nucleotide sequence encoding a cis-acting ribozyme that is either the same or different.

51. The vector of claim 48 wherein the first gene, second gene, or both have at their carboxy termini a cleavage signal.

52. The vector of claim 51, wherein said cleavage signal is a polyadenylation signal, a transient pause site, a strong pause site, a termination site, a near upstream (NUE), or a 3′ untranslated sequence.

53. The vector of claim 52, wherein more than one signal is used.

54. The vector of claim 48, wherein said first cis-acting ribozyme or the second cis-acting ribozyme or both are derived from satellite or viroid RNA.

55. The vector of claim 54, wherein said cis-acting ribozyme is derived from satellite RNA of Tobacco Ringspot Virus or derived from satellite RNA of Arabis mosaic virus.

56. The vector of claim 48, wherein said first promoter is constitutive and said second promoter is inducible.

57. The vector of claim 48, wherein said first gene is different from said second gene.

58. The vector of claim 57, wherein said first gene is a dominant negative transgene and the second gene is a gene that when expressed the expression product can convert the dominant negative transgene into a functional gene.

59. The vector of claim 57, wherein said first gene is a proenzyme and said second gene's expression product converts the proenzyme to an active enzyme.

60. The vector of claim 57, wherein said first gene encodes for a protein in which at least one amino acid of said protein is capable of being phosphorylated and said second gene encodes for a kinase capable of phosphorylating said amino acid of said protein.

61. The vector of claim 57, wherein said first gene encodes for a first protein which comprises at least one phosphorylated amino acid and said second gene encodes for a protein phosphatase capable of dephosphorylating said amino acid of said first protein.

62. A host cell comprising a vector that comprises:
(a) a first transcription unit capable of producing a first RNA transcript of a predetermined size, wherein said first transcription unit comprises a first promoter operably linked to a nucleotide sequence comprising a transcribed region such that the transcription of said transcribed region is controlled by said first promoter, wherein said transcribed region comprises a region that encodes for a first gene, and a first non-coding region downstream of said region encoding for said first gene, wherein said first non-coding region comprises a nucleotide sequence encoding a cis-acting ribozyme; and
(b) a second transcription unit capable of producing a second RNA transcript of a predetermined size, wherein said second transcription unit comprises a second promoter operably linked to a nucleotide sequence comprising a transcribed region such that the transcription of said transcribed region is controlled by said second promoter, wherein said transcribed region comprises a region that encodes for a second gene, and a second non-coding region downstream of said region encoding for said second gene, wherein said second non-coding region comprises a nucleotide sequence encoding a cis-acting ribozyme.

63. The host cell of claim 62, wherein the first gene, second gene, or both have at their carboxy termini a cleavage signal.

64. A method of making a transgenic animal comprising inserting into the genome of said animal a vector comprising:
(a) a first transcription unit capable of producing a first RNA transcript of a predetermined size, wherein said first transcription unit comprises a first promoter operably linked to a nucleotide sequence comprising a transcribed region such that the transcription of said transcribed region is controlled by said first promoter, wherein said transcribed region comprises a region that encodes for a first gene, and a first non-coding region downstream of said region encoding for said first gene, wherein said first non-coding region comprises a nucleotide sequence encoding a cis-acting ribozyme; and
(b) a second transcription unit capable of producing a second RNA transcript of a predetermined size, wherein said second transcription unit comprises a second promoter operably linked to a nucleotide sequence comprising a transcribed region such that the transcription of said transcribed region is controlled by said second promoter, wherein said transcribed region comprises a region that encodes for a second gene, and a second non-coding region downstream of said region encoding for said second gene, wherein said second non-coding region comprises a nucleotide sequence encoding a cis-acting ribozyme.

65. The method of claim 64, wherein the first gene, second gene, or both have at their carboxy termini a cleavage signal.

66. The method of claim 64, wherein said vector is inserted into the genome of the germline of said animal.

67. The method of claim 64, wherein said vector is inserted into the genome of an unfertilized or fertilized egg of said animal.

68. The method of claim 64, wherein said vector is inserted into the genome of an embryo of said animal.

69. The method of claim 64, wherein said vector is inserted into the genome of a cell located in the uterus of said animal.

70. A transgenic non-human animal comprising a vector which comprises:
(a) a first transcription unit capable of producing a first RNA transcript of a predetermined size, wherein said first transcription unit comprises a first promoter operably linked to a nucleotide sequence comprising a transcribed region such that the transcription of said transcribed region is controlled by said first promoter, wherein said transcribed region comprises a region that encodes for a first gene, and a first non-coding region downstream of said region encoding for said first gene, wherein said first non-coding region comprises a nucleotide sequence encoding a cis-acting ribozyme; and
(b) a second transcription unit capable of producing a second RNA transcript of a predetermined size, wherein said second transcription unit comprises a second promoter operably linked to a nucleotide sequence comprising a transcribed region such that the transcription of said transcribed region is controlled by said second promoter, wherein said transcribed region comprises a region that encodes for a second gene, and a second non-coding region downstream of said region encoding for said second gene, wherein said second non-coding region comprises a nucleotide sequence encoding a cis-acting ribozyme.

71. The transgenic non-human animal of claim 70, wherein the first gene, second gene, or both have at their carboxy termini a cleavage signal.

72. A two vector retrovirus production system comprising:
(a) a first vector comprising a nucleotide sequence encoding a payload and a first promoter that controls transcription of said payload; and
(b) a second vector comprising:
(i) a nucleotide sequence encoding a structural gene and a second promoter which controls transcription of said structural gene; and
(ii) a nucleotide sequence encoding a non-structural gene and a third promoter which controls transcription of said non-structural gene, wherein said nucleotide sequence encoding said structural gene and said nucleotide sequence encoding said non-structural gene are separated by a nucleotide sequence encoding a cis-acting ribozyme.

73. The retrovirus production system of claim 72, wherein the first, second, and third promoters are the same or are different.

74. The retrovirus production system of claim 72, wherein the payload is selected from the group consisting of an antisense molecule, a RNA decoy, a transdominant mutant, a toxin, a single-chain antibody (scAb) directed to a viral structural protein, a siRNA, and a ribozyme.

75. The retrovirus production system of claim 72, wherein said structural gene is selected from the group consisting of gag, a gag-pol precursor, pro, reverse transcriptase (RT), integrase (In) and env.

76. The retrovirus production system of claim 72, wherein said non-structural gene is selected from the group consisting of tat, rev, nef, vpr, vpu, and vif.

77. A two vector retrovirus production system comprising:
(a) a first vector comprising a nucleotide sequence encoding a payload and a first promoter that controls transcription of said payload; and
(b) a second vector comprising
(i) a nucleotide sequence encoding a structural gene and a second promoter that controls transcription of said structural gene,
(ii) a nucleotide sequence encoding a non-structural gene and a third promoter that controls transcription of said non-structural gene, and
(iii) a nucleotide sequence encoding an envelope gene and a fourth promoter that controls transcription of said envelope gene, wherein each of the nucleotide sequences encoding the three genes are separated by a nucleotide sequence encoding a cis-ribozyme.

78. The retrovirus production system of claim 77, wherein the first, second, third, and fourth promoters are the same or are different.

79. The retrovirus production system of claim 77, wherein the payload is selected from the group consisting of an antisense molecule, a RNA decoy, a transdominant mutant, a toxin, a single-chain antibody (scAb) directed to a viral structural protein, a siRNA, and a ribozyme.

80. The retrovirus production system of claim 77, wherein said structural gene is selected from the group consisting of gag, a gag-pol precursor, pro, reverse transcriptase (RT), integrase (In) and env.

81. The retrovirus production system of claim 77, wherein said non-structural gene is selected from the group consisting of tat, rev, nef, vpr, vpu, and vif.

82. A method of producing a retrovirus comprising contacting a cell with a two vector retrovirus production system comprising:
(a) a first vector comprising a nucleotide sequence encoding a payload and a first promoter that controls transcription of said payload; and
(b) a second vector comprising a nucleotide sequence encoding a structural gene and a second promoter that controls transcription of said structural gene, a nucleotide sequence encoding a non-structural gene and a third promoter that controls transcription of said non-structural gene, wherein said nucleotide sequence encoding said structural gene and said nucleotide sequence encoding said non-structural gene are separated by a nucleotide sequence encoding a cis-acting ribozyme.

83. A method of producing a retrovirus comprising contacting a cell with a two vector retrovirus production system comprising:
(a) a first vector comprising a nucleotide sequence encoding a payload and a first promoter that controls transcription of said payload; and
(b) a second vector comprising a nucleotide sequence encoding a structural gene and a second promoter that controls transcription of said structural gene, a nucleotide sequence encoding a non-structural gene and a third promoter that controls transcription of said non-structural gene, and a nucleotide sequence encoding an envelope gene and a fourth promoter that controls transcription of said envelope gene, wherein each of the nucleotide sequences encoding the three genes are separated by a nucleotide sequence encoding a cis-ribozyme.


SEQUENCE LISTING (1 sequence)

SEQ ID NO: 1 - DNA, 55 bp, Comoviridae Nepovirus
cctgtcaccggatgtgctttccggtctgatgagtccgtgaggacgaaacaggact